SEQUENCE LISTING (14 sequences)

SEQ ID NO: 1 - DNA, 40 bp, artificial sequence
  cpg free at rich variant of t21 promoter preamble optimized for minimum immune system stimulation
tcagtttttcatgtttttcatgtttttcatgtttttcaca

SEQ ID NO: 2 - DNA, 836 bp, artificial sequence
  cpg free at rich repeat of SEQ ID #1
tcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgtttttcacatcagtttttcatgtttttcatgtttttcatgttttt

SEQ ID NO: 3 - DNA, 400 bp, artificial sequence
  cpg free at rich sequence - expression stabilizer
acctaggtctagctttttttaattttcaaaaatcatacaattacccattcatacaatcctctccatttcaactaaactacttcaccttctactagacttactcttgtcttcctaatccaatatacctaaatattcactttctataataaccctctataaaataatcttaacctctcccaaaacttaataacaattttctccaaatctctcactcctaccactttcacatctacattccaccacacttcccctcataattaaacctaactaactttaccactatcaacatacattcaccccccaccctcaaccacctaccatactcatttaacatactactcaccacttccctaaacacatatcactcatcaatttatttttttcaccaagaggatctttc

SEQ ID NO: 4 - DNA, 400 bp, artificial sequence
  cpg free at rich sequence - expression stabilizer
tccaattttttaataacaccacccctcatctaccccaaactactgagaatctaattatatctacattcacttacatatccatctccaccctccctcaatagtctctacaccagatgttgtcttatattgtttacctttcatacactttcatatcttacaacctttccatcctatccaaacaaattatcacaccatctaactaacactgatagaaccaatacttatcacttctcactcatacaaaccaattccaacaaaaaaccaccatcacacctaccaatccctaacatacccccctcaatctaatatattctttaatataactttaaatatcccaaatactccttccttataccctaaacagtgttaatagtcatctgtcccacctttttttcccata

SEQ ID NO: 5 - DNA, 1168 bp, artificial sequence
  Derived from E. coli Kanamycin resistance gene modified for minimum immune system stimulation
tagtcaccctccaacaccagctgactctccctgactggcttgtctgctccaggcatcagcttacagacaagctgtgaagtctcctggagctgcatgtgtcagaggttttcacagtcatcacagaaactggctagactaaagggcctagtgatactcctatttttataggttaatgtcatgataataatggtttcttagaagtcaggtggcacttttctgggaaatgtgctctgaacccctatttgtttatttttctaaatacattcaaatatgtatcagctcatgagacaataaccctgataaatgcttcaataatcctcagtaatacaaggggtgttaggtaccaatgagccatattcaaagggaaacttcttgctctaggccaagattaaattccaacatggatgctgatttatatgggtataaatgggctagagataatgttgggcaatcaggtgcaacaatctatagattgtatgggaagccagatgctccagagttgtttctgaaacatggcaaaggtagtgttgccaatgatgttacagatgagatggtcagactaaactggctgacagaatttatgcctcttcctaccatcaagcattttatcaggactcctgatgatgcatggttactcaccactgctatccctgggaaaacagcattccaggtattagaagaatatcctgattcaggtgaaaatattgttgatgctctggcagtgttcctgaggagattgcattctattcctgtttgtaattgtccttttaacagtgatagagtatttagactggctcaggcacaatcaagaatgaataatggtttggttgatgctagtgattttgatgatgagaggaatggctggcctgttgaacaagtctggaaagaaatgcataagcttttgccattctcacaggattcagttgtcactcatggtgatttctcacttgataaccttatttttgatgaggggaaattaataggttgtattgatgttggaagagttggaatagcagacagataccaggatcttgccatcctatggaactgccttggtgagttttctccttcattacagaagaggctttttcaaaaatatggtattgataatcctgatatgaataaattgcagtttcatttgatgctggatgagtttttctaatcagaa

SEQ ID NO: 6 - DNA, 918 bp, artificial sequence
  Derived from E. coli Kanamycin resistance gene modified for minimum immune system stimulation
atgagccatattcaaagggaaacttcttgctctaggccaagattaaattccaacatggatgctgatttatatgggtataaatgggctagagataatgttgggcaatcaggtgcaacaatctatagattgtatgggaagccagatgctccagagttgtttctgaaacatggcaaaggtagtgttgccaatgatgttacagatgagatggtcagactaaactggctgacagaatttatgcctcttcctaccatcaagcattttatcaggactcctgatgatgcatggttactcaccactgctatccctgggaaaacagcattccaggtattagaagaatatcctgattcaggtgaaaatattgttgatgctctggcagtgttcctgaggagattgcattctattcctgtttgtaattgtccttttaacagtgatagagtatttagactggctcaggcacaatcaagaatgaataatggtttggttgatgctagtgattttgatgatgagaggaatggctggcctgttgaacaagtctggaaagaaatgcataagcttttgccattctcacaggattcagttgtcactcatggtgatttctcacttgataaccttatttttgatgaggggaaattaataggttgtattgatgttggaagagttggaatagcagacagataccaggatcttgccatcctatggaactgccttggtgagttttctccttcattacagaagaggctttttcaaaaatatggtattgataatcctgatatgaataaattgcagtttcatttgatgctggatgagtttttctaatcagaattggttaattggttgtaacactggcaaccagtgagtgtgggtcttgcagtatcattgcagcactggggccagatggtaagccctcctgtatcatag

SEQ ID NO: 7 - DNA, 534 bp, artificial sequence
  gentamycin resistance gene modified for minimum immune system stimulation
atgctgagatcttctaatgatgtgactcagcagggttctagaccaaaaactaaactgggtggttcttctatgggtattattagaacttgtagactgggtccagatcaggtgaaatctatgagagcagcactggatctgtttggtagagaatttggtgatgtggcaacttattctcagcatcagccagattctgattatctgggtaatctgctgagatctaaaacttttattgcactggcagcatttgatcaggaagcagtggtgggtgcactggcagcatatgtgctgccaagatttgaacagccaagatctgaaatttatatttatgatctggcagtgtctggtgaacatagaagacagggtattgcaactgcactgattaatctgctgaaacatgaagcaaatgcactgggtgcatatgtgatttatgtgcaggcagattatggtgatgatccagcagtggcactgtatactaaactgggtattagagaagaagtgatgcattttgatattgatccatctactgcaacttga

SEQ ID NO: 8 - DNA, 534 bp, artificial sequence
  gentamycin, kanamyicin & tobramycin resistance optimized for minimal immune system stimulation
atggatactactcaggtgactctgattcataaaattctggcagcagcagatgaaagaaacctgccactgtggattggtggtggttgggcaattgatgcaagactgggtagagtgactagaaaacatgatgatattgatctgacttttccaggtgaaagaagaggtgaactggaagcaattgtggaaatgctgggtggtagagtgatggaagaactggattatggttttctggcagaaattggtgatgaactgctggattgtgaaccagcatggtgggcagatgaagcatatgaaattgcagaagcaccacagggtagctgcccagaagcagcagaaggtgtgattgcaggtagaccagtgagatgcaacagctgggaagcaattatttgggattatttttattatgcagatgaagtgccaccagtggattggccaactaaacatattgaaagctatagactggcatgcactagcctgggtgcagaaaaagtggaagtgctgagagcagcatttagaagcagatatgcagcatga

SEQ ID NO: 9 - DNA, 861 bp, artificial sequence
  ampicillin resistance optimized for minimal immune system stimulation
atgtctattcagcattttagagtggcactgattccattttttgcagcattttgcctgccagtgtttgcacatccagaaactctggtgaaagtgaaagatgcagaagatcagctgggtgcaagagtgggttatattgaactggatctgaactctggtaaaattctggaatcttttagaccagaagaaagatttccaatgatgtctacttttaaagtgctgctgtgtggtgcagtgctgtctagaattgatgcaggtcaggaacagctgggtagaagaattcattattctcagaatgatctggtggaatattctccagtgactgaaaaacatctgactgatggtatgactgtgagagaactgtgctctgcagcaattactatgtctgataacactgcagcaaacctgctgctgactactattggtggtccaaaagaactgactgcatttctgcataacatgggtgatcatgtgactagactggatagatgggaaccagaactgaatgaagcaattccaaatgatgaaagagatactactatgccagtggcaatggcaactactctgagaaaactgctgactggtgaactgctgactctggcatctagacagcagctgattgattggatggaagcagataaagtggcaggtccactgctgagatctgcactgccagcaggttggtttattgcagataaatctggtgcaggtgaaagaggttctagaggtattattgcagcactgggtccagatggtaaaccatctagaattgtggtgatttatactactggttctcaggcaactatggatgaaagaaacagacagattgcagaaattggtgcatctctgattaaacattggtga

SEQ ID NO: 10 - DNA, 798 bp, artificial sequence
  Neomycin resistance optimized for minimal immune system stimulation
atgggtattgaacaggatggtctgcatgcaggttctccagcagcatgggtggaaagactgtttggttatgattgggcacagcagactattggttgctctgatgcagcagtgtttagactgtctgcacagggtagaccagtgctgtttgtgaaaactgatctgtctggtgcactgaatgaactgcaggatgaagcagcaagactgtcttggctggcaactactggtgtgccatgtgcagcagtgctggatgtggtgactgaagcaggtagagattggctgctgctgggtgaagtgccaggtcaggatctgctgtcttctcatctggcaccagcagaaaaagtgtctattatggcagatgcaatgagaagactgcatactctggatccagcaacttgcccatttgatcatcaggcaaaacatagaattgaaagagcaagaactagaatggaagcaggtctggtggatcaggatgatctggatgaagaacatcagggtctggcaccagcagaactgtttgcaagactgaaagcaagaatgccagatggtgaagatctggtggtgactcatggtgatgcatgcctgccaaacattatggtggaaaatggtagattttctggttttattgattgtggtagactgggtgtggcagatagatatcaggatattgcactggcaactagagatattgcagaagaactgggtggtgaatgggcagatagatttctggtgctgtatggtattgcagcaccagattctcagagaattgcattttatagactgctggatgaatttttttga

SEQ ID NO: 11 - DNA, 392 bp, artificial sequence
  R6K origin of replication optimized for minimal immune system stimulation
ccatgtcagccattaagtgttcctgtgtcactcaaaattgctttgagaggctctaagggcttctcagtacattacatccctggcttgttgtccacaaccattaaaccttaaaagctttaaaagccttatatattcttttttttcttataaaacttaaaaccttagaggctatttaagttgctgatttatattaattttattgttcaaacatgagagcttagtacatgaaacatgagagcttagtacattagccatgagagcttagtacattagccatgagggtttagttcattaaacatgagagcttagtacattaaacatgagagcttagtacatgaaacatgagagcttagtacatactatcaacaggttgaactgctgatcttcagatc

SEQ ID NO: 12 - DNA, 259 bp, artificial sequence
  R6K origin of replication optimized for minimal immune system stimulation - short
aaaccttaaaacctttaaaagccttatatattcttttttttcttataaaacttaaaaccttagaggctatttaagttgctgatttatattaattttattgttcaaacatgagagcttagtacatgaaacatgagagcttagtacattagccatgagagcttagtacattagccatgagggtttagttcattaaacatgagagcttagtacattaaacatgagagcttagtacatactatcaacaggttgaactgctgat

SEQ ID NO: 13 - DNA, 389 bp, artificial sequence
  R6K origin of replication optimized for minimal immune system stimulation
catgtcagcagttaagtgttcctgtgtcactcaaaattgctttgagaggctctaagggcttctcagtgagttacatccctggcttgttgtccacaacagttaaaccttaaaagctttaaaagccttatatattcttttttttcttataaaacttaaaaccttagaggctatttaagttgctgatttatattaattttattgttcaaacatgagagcttagtaagtgaaacatgagagcttagtaagttagccatgagagcttagtaagttagccatgagggtttagttagttaaacatgagagcttagtaagttaaacatgagagcttagtaagtgaaacatgagagcttagtaagtactatcaacaggttgaactgctgatcttcagc

SEQ ID NO: 14 - DNA, 535 bp, artificial sequence
  pBR322 origin of replication optimized for minimal immune system stimulation
atcttcttgagatcctttttttctgagagtaatctgctgcttgcaaacaaaaaaaccacagctaccagaggtggtttgtttgcaggatcaagagctaccaactctttttcagaaggtaactggcttcagcagagagcagataccaaatactgtccttctagtgtagcagtagttaggccaccacttcaagaactctgtagcacagcctacatacctagctctgctaatcctgttaccagtggctgctgccagtggagataagtagtgtcttacagggttggactcaagaagatagttacaggataaggagcagaggtagggctgaaaggggggttagtgcacacagcccagcttggagagaaagacctacacagaactgagatacctacagagtgagctatgagaaagagccaagcttccagaagggagaaaggaggacaggtatcaggtaagaggcagggtaggaacaggagagagcaagagggagcttccagggggaaaagcctggtatctttatagtcctgtagggtttagc

(19) United States
(12) Patent Application Publication
(10) Pub. No.: US 2012/0213848 A1
(43) Pub. Date: Aug. 23, 2012

(54) Treatment of Infection Using Single Chain Antibody Gene Therapy

(76) Inventor: Brian P. Hanley, Davis, CA (US)

(21) Appl. No.: 13/298,251

(22) Filed: Nov. 16, 2011

Related U.S. Application Data
(60) Provisional application No. 61/414,146, filed on Nov. 16, 2010.

(51) Int. Cl. A61K 9/48; A61K 9/14; C12N 15/63; A61P 31/06; A61P 29/00; A61P 37/06; A61P 31/18; A61P 31/04; A61K 48/00; A61P 31/00

(52) U.S. Cl. 424/463; 514/44 R; 424/499; 424/400; 435/320.1; 424/278.1

(57) ABSTRACT

A method for treating HIV and other intra-cellular parasites and toxins using intrabodies delivered to leukocytes.

### RELATED APPLICATIONS

This application claims priority from the U.S. provisional application with Ser. No. 61/414,146, which was filed on Nov. 16, 2010. The disclosure of that provisional application is incorporated herein as if set out in full. This application is further related to a U.S. patent application filed on an even date herewith: “Methods and Compositions for Gene Therapy and GHRH Therapy”, filed as a U.S. Nonprovisional Patent Application (Attorney docket 244.12).

### REFERENCE TO SEQUENCE LISTING, COMPUTER PROGRAM, OR COMPACT DISK

The instant application contains a Sequence Listing which has been submitted in ASCII format via EFS-Web and is hereby incorporated by reference in its entirety. Said ASCII copy, created on Sep. 16, 2011, is named “Sequence listing.txt” and is 12,178 bytes in size.

### BACKGROUND

1. Field of the Invention

The present application relates generally to treating disease, and more particularly to a method and device for combatting HIV and other infections and intracellular toxins in humans and animals that employs a single chain antibody.

2. Background of the Invention

Human antibodies comprise a light chain and a heavy chain that together make up the epitope binding region of the antibody. An epitope is the part of an antigen that an antibody recognizes and binds to. The epitope binding region of an antibody matches various characteristics of a corresponding epitope, usually with high specificity.

Normal antibodies in humans and many mammals (such as mice) have a rough Y shape, with each short “arm” of the Y having an identical epitope binding region that binds to an epitope. The longer “tail” of the Y is a chain that is constant within a species and is called the Fc chain. Further detail regarding the Fc chain and antibodies in general may be found in immunology texts such as Cellular and Molecular Immunology, Saunders 7^{th} Edition, which is incorporated herein by reference as if set out in full.

Camelid antibodies differ significantly from human and mouse antibodies in that they have an epitope binding region made from a single chain (Hamers-Casterman et al, 1993). In human, and most mammalian antibodies, the epitope binding region is made from two separately coded chains (the above reference light and heavy chains) that must be assembled later. This two chain nature of human antibody epitope binding regions makes them difficult to work with, synthesize, or modify by standard molecular biology methods. Camelid antibodies, on the other hand, are quite amenable to synthesis or modification by standard molecular biology methods because the single chain epitope binding region will form properly when coded into a plasmid. Additionally, an Fc chain can be grafted onto the active site of a camelid originated DNA coding for an epitope binding region. When the Fc chain is of human sequence, this grafting method is known as humanization. This grafting of a human Fc chain onto an epitope binding region sequence allows the antibody to be present in the human body without causing an immune system response. An alternative name for the epitope binding region is the complementarity determining region (CDR).

The observation that viruses are generally deactivated by a single antibody was published by Dulbecco in 1956. Dulbecco offered speculations as to the mechanism of deactivation, such as antibodies changing the viral geometry by adhesion, an idea that was generally accepted. Recently, another mechanism of viral deactivation has been postulated and proven. It is now known that cells produce a factor referred to as TRIM21, which has the highest known affinity for the Fc chain of antibodies (Mallery et al, 2010). TRIM21 is a protein found in the cytosol internal to cells, and by binding, it causes ubiquitination of antigens that are bound by an antibody. This, in turn, results in the antibody-antigen complex being routed to the proteasome of the cell for destruction. This mechanism has recently been identified as the reason why viruses can be deactivated by a single antibody. This mechanism is more complete and offers ways to control the HIV virus and other intracellular pathogens and toxins.

The HIV virus is a lentivirus, and has an internal protein capsid that covers the RNA material at the center of the virus. Surrounding this capsid is an envelope made from the membrane of whatever cell the HIV virus buds from. Floating in the membrane envelope of the viral cell is GP41 and GP120 (Zhu et al, 2006). GP120 is a flexible protein that binds to a number of things, but its primary role in HIV infection is believed to be that it binds to the CD4 T-cell receptor and also to the CXCR4 receptor.

In classical infection theory, after attachment to a CD4 or CXCR4 or CCR5 bearing leukocyte, the virus merges its membrane into the membrane of the host cell and releases its internal capsid into cytosol there (Swan et al, 2005). A more recent theory is that the virus is engulfed by endocytosis and makes its way into the cytosol inside a vesicle (Miyauchi et al, 2009). The capsid and its contents are released into cytosol from the vesicle when deep inside the cell.

A more controversial proposed mechanism for infection by HIV has been hypothesized by the Applicant, and has been published by others. This hypothesis states that in addition to the above discussed classical mechanisms, HIV can be transmitted directly, cell to cell, by exchange of membrane with HIV capsid attached to the inside. The Applicant submitted an unpublished letter to Science regarding this in 2009, noting that unless protocols for infection of monkeys in primate studies for HIV vaccines were altered to employ this method, that all such studies would give erroneous results. Direct exchange of membrane with GP120 proteins, cell-to-cell, has been shown, and it follows that the small capsids that are sitting next to the membrane will also be exchanged. Multiple studies have now shown this does occur (Hübner, et al, 2009, Chen et al, 2007) however, this idea has only very slowly to filtered into mainstream thought and analysis. Additionally, a decades-old unpublished observation by an HIV virus culture specialist states that inoculating a new crop of expanded leukocytes with living infected leukocytes was the fastest way to achieve high titers of virus—days faster than inoculating with HIV virus itself, regardless of the titer of purified virus used to inoculate. This unpublished observation strongly suggests that infected cells played a direct role in infection.

This direct cell-to-cell infection hypothesis also correlates with HIV vaccine trials sometimes resulting in slightly higher infection rates in vaccinated versus unvaccinated population. This difference in infection rates can be explained by the above discussed cell-to-cell infection hypothesis because vaccination will sensitize the infectee host immune system to infected T-cells (which are presenting GP120 and other HIV peptides), and thus the infectee immune system will attack the infected T-cells, increasingly the likelihood and duration of membrane-to-membrane contact, thus slightly improving the odds of transfer of HIV by a viral synapse. Additionally, sensitized attack by one set of T-cells and macrophages on foreign T cells bearing HIV antigens can result in destruction of that cell and endocytosis of the cell's components, thus ensuring transmission of very large quantities of HIV virus into the attacking cell.

Currently there are many projects attempting to create a vaccine against HIV. These methods depend on developing natural antibodies to HIV antigens. Most of them concentrate on the development of antibodies to sections of GP120 in the theory that by binding to GP120 successfully, virus will be blocked from entering cells. This idea is based on the notion that a primary mechanism of deactivation of viruses is that antibodies prevent them from entering cells by occupying their binding sites. These methods have proven to be unsuccessful to date with HIV. The greatest single reason for their lack of success is that direct cell-to-cell transmission without release into cytosol is the primary mode of infection as discussed above.

A number of patents exist for various forms of single-chain antibodies. In patent literature, these are sometimes known as nanobodies. Introducing these and other antibodies into cytosol via DNA has been worked on by others and the term intrabody has been coined for it (Chen et al 1994, Lo et al, 2008).

Some efforts have been made to treat HIV/AIDS using intrabodies (intracellular antibodies) (Mhashilkar et al, 1995; Marasco et al, 1999; Legastelois & Desgranges, 2000; Swan et al, 2005; Swan et al 2006). These have concentrated on using human or murine origin antibodies. In some cases, these antibodies have been engineered from a dual chain into a single-chain format. This re-engineering has been done by development of difficult to execute methodologies for molecular biology manipulation using designed linker peptides between the heavy and light chain fragments to produce artificial, single chain antibody sequences from dual chain sources.

A relatively new area of immunology relating to single-chain antibodies depends upon the characteristics of camelid antibodies (Hamers-Casterman et al, 1993). These antibodies derive from camels, alpacas, llamas, guanacos and vicunas. These antibodies are different than human, mouse, rabbit and many other common mammalian antibodies. A subset of camelid antibodies have a single chain for their epitope binding region, (the heavy chain) and are entirely lacking the light chain of most antibodies. Having a single chain for coding the epitope binding region is very convenient for molecular biology. Primers can be created for the constant region of the antibody chain, and using PCR methods these primers can then be used to copy the rest of the chain from active immune cells producing antibody. That means that it is possible to collect the DNA coding for a camelid antibody's epitope binding region from an animal by collecting a blood sample. That avoids cost and other problems associated with sacrificing the animal, harvesting the spleen, fusing splenic cells to create hybridomas specific to the species and finding a clone that will stably produce the antibody.

In addition, the epitope binding region of camelid antibodies can then be used in phage display systems to improve or lower affinity to an antigen as needed. There are phage display systems that use dual chain antibody, but they are slow to produce results and tricky to use (Gao et al, 1999).

The epitope binding regions of camelid antibodies are quite small. By molecular biology methods, the coding DNA can be spliced to the DNA coding for an Fc chain of a human (or other animal) antibody. When a human Fc chain is spliced to it, this process is known as humanization. The result is a single-chain, small antibody that is useful therapeutically. For the purposes of this application, the Fc chain coding sequence employed would generally not contain a secretion sequence.

An advantage of using molecular biology methods to create functional antibodies (from any source, camelid, or otherwise) is that the DNA can be stored in libraries, both in a freezer and in-silico. The single-chain antibodies can be produced by fermentation. The sequences may stored in computers for later synthesis in case the DNA sequence in storage is lost.

The treatment disclosed in this application utilizes these advantages nanobodies and intrabodies, and adds direct intracellular delivery of the antibody into cells with other features to maximize effectiveness of the treatment.

Plasmids, minimal DNA cassettes, and other forms of DNA have been used for some time to transfect cells. It is known that CpG (cytosine, phosphate sugar, guanine) sequences (going 5′ to 3′ in direction) are immunogenic (Brazolot-Millan et al, 1998). However, generating an immune response to a therapeutic protein coded for by DNA gene therapy is highly undesirable. Some researchers have found that until complete elimination of all CpG sequences, not just the highly antigenic longer motifs, the vaccine response does not disappear. Consequently, CpG sequences are a priority to completely eliminate from therapeutic DNA.

The constructs of this invention comprise one or more AT rich regions that are 5′ or 3′ to the expression cassette. These AT rich regions help to improve the expression in animal cells of the gene carried by the plasmid. These AT rich regions avoid start codons in order to prevent the presence of an open reading frame (ORF). Certain sequences of AT rich regions were also developed on the basis of literature showing efficacy, but redesigned to conform with the immune system optimization of the present invention. Specific implementations of these sequences are further defined in Seq ID 1, 2, 3 and 4. These sequences contain no CpG sequences, and act to aid in stabilizing long-term expression of therapeutic genes comprising.

Heretofore, there has been no clear rationale for why it might be useful to introduce DNA coding for antibodies directly into infected cells, however, there have been some efforts in that direction (Chen et al, 1994, Rossi et al, 2007). These efforts have taken a shotgun approach, using fragments and other components. With the publication of the TRIM21 system, this rationale is laid out and the optimal design is clear.

It is thus a first object of the present application to provide a treatment in which DNA coding for antibodies is introduced directly into infected cells so that the antibodies coded by them become present in cytosol.

It is a further object of the present application to provide a treatment that employs molecular biology methods to make intrabodies from Camelid single-chain antibodies, and to put the codons for the epitope binding region of those antibodies together with a non-secreting human Fc chain into functional DNA cassetts.

It is a further object of the present application to provide a treatment that minimizes immune response by removing all CpG sequences.

It is a further object of the present application to provide a treatment that delivers the DNA cassetts on and in microbeads of optimum size directly into the lympy and peritoneal vacity and orally, thus generally avoiding the bloodstream.

It is a further object of the present application to provide a treatment that utilizes anti-adjuvant compounds such as 2,5-dimethoxy-4-iodoamphetamine (DOI) (Yu et al, 2008) to counteract the adjuvant characteristics of microbeads.

It is a further object of the present application to provide a treatment that can be used as a standalone treatment fort HIV, TB, or other intracellular parasites or toxins, and which may be used in conjunction with other treatments such HAART (highly active anti-retroviral therapy) or GHRH gene therapy.

It is a final object of the present application to provide a treatment that utilizes the advantages of nanobodies and intrabodies combined with direct intracellular delivery of the antibody into cells.

### SUMMARY OF THE INVENTION

The present application presents a treatment of infection using single chain antibody gene therapy. This treatment utilizes the advantages of nanobodies and intrabodies combined with direct intracellular delivery of the antibody into cells, as well as a variety of techniques to maximize effectiveness of the technique.

### BRIEF DESCRIPTION OF THE FIGURES

The foregoing aspects and many of the attendant advantages of the invention will become more readily appreciated as the same becomes better understood by reference to the following detailed description, when taken in conjunction with the accompanying drawings, wherein:

FIG. 1 shows a diagram of the essential components of a typical plasmid containing a mammalian promoter, an Fc chain coding region, an epitope binding coding region, and a poly-A terminator; these components could be inserted into any plasmid capable of reproduction in any host microbe.

FIG. 2 shows a diagram of the essential components of the plasmid along with AT rich regions free of CpG sequences; these AT rich regions provide for stabilization of transcription. As above, these components could be inserted into any plasmid that is capable of reproduction in any host microbe.

### DETAILED DESCRIPTION OF THE INVENTION

The treatment present by this application was created in light of the recently published knowledge that there is a receptor, TRIM21, present in cytosol. TRIM21 binds with the highest affinity yet found to the Fc chain of antibodies. The TRIM21 receptor appears to be what allows a single antibody to deactivate a virus. This treatment presented in this application utilizes TRIM21 to immunize leukocytes against infection. This treatment is specifically useful against HIV in two ways. First, cells that are transformed will be able to deactivate the HIV virus as it arrives within the cell. Second, cells which are already infected and producing HIV particles will destroy the HIV proteins that are produced in cytosol before the proteins can assemble into a virus capsid, thus controlling the effects of infection by HIV.

The treatment presented in this application provides the following advantages:
(a) The treatment provides a method of attacking viruses, bacteria and toxins inside cellular cytosol using the immune system's TRIM21 mechanism by use of molecular biology derived single chain epitope binding regions derived primarily from camelid antibody producing cells that are combined with Fc chains. This treatment may be applied to persistent viral infection such as HIV, herpes virus infections, and to persistent intracellular parasite bacteria such as Mycobacterium tuberculosis.
(b) The treatment provides, for the first time, for delivery of DNA coding for antibody and other plasmid deliverable products into sufficient T-cells and macrophages in the human body to make such treatment practical. The treatment provides a method for introducing practical antibody production in immune system cells specifically, thus immunizing them to HIV. This is accomplished by use of treatment DNA cassettes impregnated into, and/or adsorbed onto, microbeads made of a material like poly lactide-co-glycolide (PLG) or polyethylene glycol (PEG) (Tracy et al, 1999; Vila et al, 2003) mixed with other materials. For instance, materials might be introduced into microbeads for control of pH of encapsulated materials. Since T-cells cells are the target of HIV infection, and the intra-host propagators of the HIV infection, immunizing them is an important factor. Not only does it drastically lower HIV virus particles emitted from cells, but it can render infected cells virtually unharmed by continuous destruction of HIV particle components.
(c) The treatment provides, for the first time, for delivery of DNA coding for antibody and other plasmid deliverable products with minimal stimulation of the immune system by the treatment cassettes themselves.
(d) The treatment provides for counteracting the immune system adjuvant effect of microbeads by co-injection with anti-inflammatory or some compound which has an anti-adjuvant effect on the immune system.

In this treatment, the amino acid chains of one or more antibodies that bind to one or more of: HIV reverse transcriptase, p51 and/or p66; HIV trans-activator of transcription (tat); internal capsid protein, p24; and/or attachment glycoproteins, and/or Gp41 and/or Gp120; are coded into one or more chains of DNA together with their respective Fc chains into expression cassettes. Each cassette is generally made separately, although it would be possible to produce all of the cassettes from one plasmid by use of the CHYSEL system (de Felipe, 2004).

The one or more chains of DNA cassettes (“the DNA”) are introduced into leukocytes in vivo or in vitro. The DNA contains one or more promoters upstream of the genes for the antibody peptide chains to activate production of the antibodies coded for by the DNA introduced into the cell.

Consequently, when HIV viruses invade a cell that contains antibodies in cytosol to these components of the HIV virus, those components will be routed to the proteasome, thus preventing the virus from infecting the cell. In the case of cells that are already infected by the HIV virus, when HIV proteins appear in the cell, they will be bound by antibodies immediately and destroyed by the proteasome, thus minimizing the effect of the HIV virus on the cell.

In accordance with the preferred embodiment of the invention, the treatment makes use of one or more plasmids with DNA coding for humanized single-chain antibodies (which will be considered a type of antibody herein) to one or more HIV components which are free of CpG sequences. The invention will comprise a DNA plasmid construct containing a bacterial plasmid origin of replication that does not contain CpG sequences, a bacterial antibiotic resistance gene that does not contain CpG sequences, a high activity general purpose promoter containing no CpG sequences, a humanized synthetic antibody gene composed of an Fc-chain sequence and an epitope binding region sequence for an antigen which contains no CpG sequences, a poly-A termination sequence containing no CpG sequences, and one or more AT rich regions between the bacterial section and the mammalian expression half of the plasmid which also contain no CpG sequences. This construct is delivered into leukocyte cells together with DOI at a dose generally above 0.5 micrograms per kilogram, dissolved into a water and salts medium suitable for preservation of DNA.

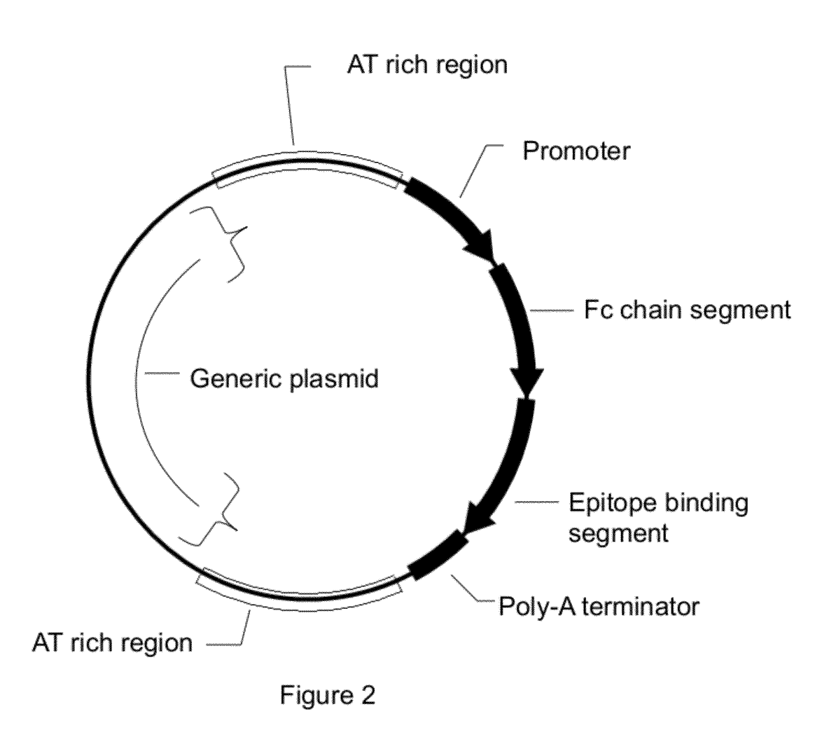

The preferred embodiment of the invention is shown in FIG. 2. In this figure, the components of the cassette are shown, with a callout for “Generic plasmid” to encompass the rest. The “Generic plasmid” section of the diagram would contain the bacterial origin of replication, linker segments and the bacterial antibiotic resistance gene.

Generally, plasmids comprised as described that code for humanized single-chain antibodies to HIV reverse transcriptase, (p51 and/or p66), HIV tat, and HIV internal capsid protein, (p24) may be present singly or together. This multiplicity of plasmids are purified, then adsorbed onto and impregnated into microbeads made of a material such as poly lactide-co-glycolide (PLG) and preferably sized close to 2 microns, this combination of plasmids and microbeads may be thought of as part of a preparation employed in the treatment. The plasmid adsorption and impregnation of microbeads should be done so as to obtain a population of all plasmid types on or within all microbeads. The purpose of the PLG microbeads is to improve selective uptake of DNA by leukocytes specifically and increase the number of plasmids delivered to each leukocyte. Each particle will deliver a significant dose of antibody coding plasmids into the cell.

As this is a gene therapy treatment, and immunogenicity of plasmids/DNA is a factor, the present application provides two separate methods for addressing this concern. First, plasmids used herein are preferably CpG (cytosine-phosphate-guanine) free to minimize immunogenicity of the preparation. Second, the preparation preferably comprises an appropriate amount of 2,5-dimethoxy-4-iodoamphetamine (DOI), generally between 100 and 400 total micrograms per treatment. The purpose of the DOI is to suppress production of TNF-alpha for the first 24 hours after injection in order to minimize immunogenicity of the injection. Other compounds as known in the art may also be useful to co-inject for short term interference with the intracellular immune system.

The above preparation may then be injected into a patient. The injections are preferably done at several locations on the body, generally avoiding blood vessels. These location on the body may preferably comprise: the intraperitoneal cavity, generally underneath the mesenteric fat pad; in several locations under the skin; and directly into lymph at several locations. It may be desirable to minimally anesthetize the patient prior to the procedure, or to co-inject a small quantity of an anesthetic such as procaine, since injection into lymph can be quite painful. Injections should not be made intramuscularly, and should generally avoid injection into subcutaneous fat or directly into blood vessels.

The number of microbeads required for treatment is quite high and thus sample calculations for dose requirements are presented below. These calculations are based on the assumption that roughly 17% of the human body by weight is leukocytes, most of which are not in blood. Based on this assumption, there are approximately 8 trillion leukocytes to target in an average human.

The Poisson distribution describes the relation between target cells and the number transfected.

P(k)=e^{-m}k/k!

In this equation, P(k) is the fraction of cells transfected by k microbeads, and m is the multiplicity of transfection (MOT). The equation can be simplified to calculate the fraction of non-transfected cells (k=0), and cells with one or more transfections given any m:

P(O)=e^{-m}

P(>O)=1−e^{-m}

Thus, an MOT of 1 (e.g. microbeads=number of cells) means that approximately 37% of the cells will not be transfected, and roughly 63% will be transfected. Depending on the fraction of cells which are desired to be transfected, the microbead count will vary. An MOT of 2 yields roughly 87% transfected. An MOT of 3 should yield roughly 95% transfection. An MOT of 5 should yield roughly 99% transfection.

The closest packing of spheres is roughly 74% of volume taken up by the spheres. For our purposes, the spheres need to be suspended in solution, and thus the fraction will generally be closer to 10% of fluid volume. Assuming a nominal microsphere size of approximately 2 microns diameter, at closest packing, roughly 6.25×10¹¹ microbeads will be present per cubic centimeter. To obtain an MOT of 2, (17×10¹² microspheres) would require approximately 27 cc of close packed microbeads. Consequently, the treatment protocol for an MOT of 2 would require a total 270 cc of 10% by volume solution. This would be divided up into the set of injections discussed above.

It can easily be seen that while an MOT of 1 or 2 could be injected in a matter of an hour to multiple locations, to attain an MOT of 5 would require 675 cc to be administered. This quantity would generally be best delivered over multiple treatment sessions.

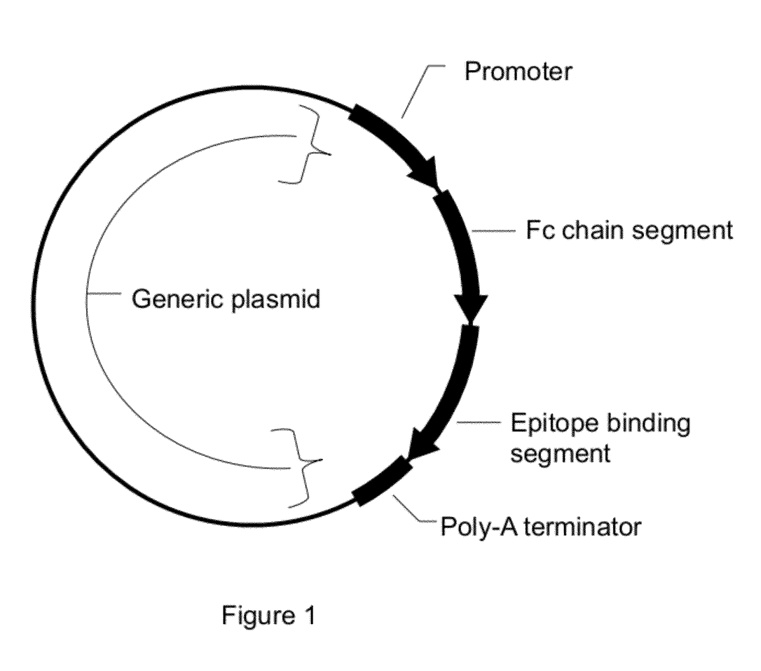

In an alternative embodiment, as shown in FIG. 1, the plasmid used for treatment could be lacking the specific AT rich regions from the cassette which help stabilize expression. The expression stabilization in this alternative could be provided by regions provided by a commercially available or later designed sequence.

In another alternative embodiment, the microbeads may contain a small quantity of an imaging agent such as Gallium-67 so that location can be tracked during and after treatment.

In an additional alternative embodiment, infusions may be made directly into a single site such as intra-peritoneal or directly into a chosen lymph site at variable rates as appropriate.

In an additional alternative embodiment DNA may be directly injected together with an anti-adjuvant such as DOI. In embodiment, the same injections or infusions would be done into the same kinds of locations as discussed above, however the volume of fluid could be less.

An alternative long-acting anti-inflammatory may be substituted for DOI, or else separate anti-inflammatory treatment could be given.

In an additional alternative embodiment the DNA employed in this treatment may be produced by direct synthesis. Such synthesis techniques are currently being developed for practical application by Vical Corporation out of San Diego for rapid production of DNA vaccines. Directly synthesized DNA may then be substituted for the plasmids above, avoiding a number of issues with production of plasmids in single celled organisms. In this embodiment, only the active nucleotides required for production of intra-cellular antibodies would be present.

In a further alternative embodiment of special interest, plasmids may be post-processed to remove all but the essential features of the expression cassettes. Other embodiments of the Applicant's invention are possible and are discussed below. This can be seen in FIGS. 1 and 2, as the “Generic plasmid” callout. By placement of restriction sites or topoisomerase sequences at the ends of the “Generic plasmid” sections, this piece can be removed from the plasmid. This post-processing can include purification to separate the therapeutic segment of the invention.

A similar strategy (not shown) removes only the bacterial origin of replication from the plasmid, instead of all elements pertaining to the bacteria. This is useful because the shorter segment can be more amenable to removal by topoisomerase, and the bacterial origin of replication has the greatest impact on copy number.

A further alternative embodiment has plasmids that may be constructed with more than one DNA cassette. Those multiple cassettes can be expressed using the CHYSEL system or something similar.

An alternative embodiment may have a different microbead material known in the art substituted for PLG or PEG.

In an alternative embodiment, leukocytes may be harvested from the patient, then transfected using conventional techniques or using the PLG microbead system. In this embodiment, after transfection with DNA the cells may be returned to the patient. In this embodiment, generally, leukocytes would be removed from blood, blood would be returned to the patient, and it would require a great many repetitions to acquire sufficient cells to make be efficacious. In a further modification of this embodiment, leukocytes could be harvested directly from lymph using a flow-through system which inoculates the lymph.

In a further alternative embodiment, the genes for the antibodies may be other than humanized camelid antibodies, and may be instead another antibody or molecularly manipulated antibody system that binds to infectious agent component molecules and has a peptide chain that TRIM21 has strong affinity for so as to route HIV component molecules, (or the molecules of some other intracellular parasite or toxin), to the proteasome for destruction.

Rather than having a simple high activity promoter, (e.g. the human cytomegalovirus promoter, or a synthetic promoter) to activate production of antibody genes, some or all of the promoters may be specific to an immune system pathway. In a further alternative promoter system, any other promoter may be used.

In another further alternative, bone marrow cells extracted from a patient may be treated directly. In this alternative embodiment, bone marrow cells would receive a high MOT, on the order of 50 to 100. The purpose of this high MOT is to deliver very high numbers of DNA cassettes into each transfected cell. In this way, when the cells divide to produce new T-cells, the new T-cells will contain sufficient DNA cassettes capable of expression for quite a few generations, and in this way maintain patient immunity.

In a further alternative embodiment, the genes for the antibodies may be inserted into a retroviral vector, injected into patients, or added to bone marrow extracted from the patient. In general, the latter method would be preferred for viral gene therapy delivery of this DNA material. After retroviral transformation, bone marrow cells may be monitored for signs of lymphoma or leukemic transformation prior to injecting them back into bone marrow.

In a similar, but different alternative, cassettes may be packaged into a non-reproductive lentiviral vector for delivery into lymph as previously described. This alternative embodiment specifically targets the roughly 30% T cells that make up leukocytes with more accuracy.

In a further alternative, microbeads bearing DNA may be delivered orally. These orally administered microbeads may also contain an anti-adjuvant material such as DOI or some other immune system counteracting compound.

The treatment disclosed by this application has application to HIV prevention and treatment as well as treatment of other viral diseases and primarily intracellular toxic conditions. The method defined herein may also be applied to treatment of diseases such as Mycobacterium tuberculosis (“TB”). Humanized single-chain antibody epitope binding sites to cord factor, mycolic acids and other TB components could be delivered to TB infected hosts. Similarly, if vectors can be developed to deliver plasmid into the liver, cells could be immunized intracellularly to hepatitis C. With vectors to deliver plasmid into herpes virus lesions, the method may be useable to control such lesions as well.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

I claim:

1. A method for intracellular immunization against an infection or toxin using a synthetic intracellular antibody, the method comprising:
a. providing an antibody production DNA cassette comprising:
i. a first DNA sequence coding for an Fc chain sequence gene;
ii. a second DNA sequence coding for a single-chain antibody epitope binding region for an antigen that is operationally linked to said first DNA sequence;
iii. a promoter, operationally linked to said first DNA sequence and second DNA sequence so as to control transcription of the synthetic intracellular antibody;
iv. a polyA_signal operationally linked to said first DNA sequence and second DNA sequence so as to terminate transcription; and
v. wherein the DNA cassette is free of CpG sequences;
b. delivering said DNA cassette into a mammalian cell; and
c. producing said antibody inside the cell by normal cellular peptide synthesis under control of said promoter, wherein, subsequent to said delivery, a synthetic single-chain antibody is produced inside the cell, and is present in the cell's cytosol and available for binding by TRIM21.

2. The method according to claim 1 further comprising a plurality of DNA cassettes coding for synthetic single-chain antibodies to different antigens.

3. The method of claim 1 wherein the DNA cassette is flanked, 5′ and 3′ by at least one AT rich region of length 40 to 1000 nucleotides.

4. The method of claim 3 wherein said AT rich region has the sequence of SEQ ID 1, 2, 3, or 4, wherein said sequences may be repeated.

5. The method according to claim 1 wherein the DNA cassette is inserted into a plasmid.

6. The method of claim 5 wherein the plasmid and its contents are free of CpG sequences.

7. The method of claim 6 wherein the plasmid contains an antibiotic resistance gene having the sequence of SEQ ID 5, 6, 7, 8, 9, or 10.

8. The method of claim 6, wherein the plasmid has a rep_origin having the sequence of SEQ ID 11, 12, 13 or 14.

9. The method according to claim 1 wherein said delivering step further comprises the addition of a compound that is at least one of an anti-inflammatory and an anti-adjuvant that suppresses immune responses.

10. The method according to claim 9 wherein said compound is DOI.

11. The method of claim 1 further comprising a local anesthetic with the is co-injected.

12. The method according to claim 1 wherein said DNA cassette is adsorbed onto or impregnated within microbeads.

13. The method according to claim 12 wherein said microbeads are approximately of a nominal 2 microns in diameter.

14. The method according to claim 12 wherein said microbeads comprise at least one of poly(lactide-co-glycolide) (PLG), and polyethelyne glycol (PEG).

15. The method according to claim 1 wherein said first DNA sequence codes for a human compatible Fc chain lacking a secretion sequence.

16. The method according to claim 1, wherein said antigen is from a microbe.

17. The method according to claim 16, wherein said antigen is at least one HIV protein selected from the group consisting of:
a. HIV reverse transcriptase (p51, p66);
b. HIV transcription activator, (tat);
c. HIV internal capsid protein, (p24); and
d. HIV attachment glycoprotiens (Gp41, Gp120).

18. The method of claim 1, wherein said delivery is accomplished by injection into the lymphatic system.

19. The method of claim 1, wherein said delivery is accomplished by injection into the intraperitoneal space.

20. The method of claim 1, wherein said delivery is accomplished by injection under the dermis.

21. The method of claim 12, wherein said delivery is accomplished by oral administration.

22. The method of claim 12, wherein the microbeads are contained in an enteric coated capsule to improve the survival of the DNA into the intestinal tract.

23. The method according to claim 16, wherein said antigen is to components of one or more of Mycobacterium tuberculosis, M. bovis, M. Africanus or related mycobacteria, including, but not limited to, one or more of cord factor and/or mycolic acids.

24. A composition of matter for intracellular immunization against an infection or toxin, the composition comprising:
a. a synthetic antibody coding cassette comprising DNA coding for an antibody epitope binding region gene derived from single chain camelid antibodies against an antigen, operationally linked to DNA coding for a peptide with affinity to TRIM21, wherein an antibody will be produced by cellular peptide synthesis under control of a promoter, such that the antibody is produced inside a cell and becomes present in the cell's cytosol and available for binding by TRIM21.

25. A preparation comprising:
a. at least two microbeads;
b. at least one DNA cassette coding for a synthetic antibody comprising:
i. a promoter;
ii. a first DNA sequence for a peptide with TRIM21 affinity;
iii. a second DNA sequence for an antibody epitope binding region;
iv. wherein said second DNA sequence is derived from a camelid antibody by molecular biology methods;
v. a polyA_signal; and
vi. wherein said first and second DNA coding sequences are operationally linked under the control of the promoter and the polyA_signal.

26. The preparation of claim 25 wherein the DNA cassette is flanked, 5′ or 3′ by at least one AT rich region of length 40 to 1000 nucleotides.

27. The preparation of claim 26 wherein said AT rich regions have the sequence of SEQ ID 1, 2, 3, or 4, wherein said sequences may be repeated.

28. The preparation of claim 25 wherein the coding cassette and its contents are free of CpG sequences.

29. The preparation according to claim 25 further comprising the addition of a compound that is an anti-adjuvant that suppresses immune responses.

30. The preparation according to claim 29 wherein said anti-adjuvant is DOI.

31. The preparation of claim 25 further comprising a local anesthetic.

32. The preparation according to claim 25 wherein said DNA cassette is adsorbed onto or impregnated within microbeads for delivery into a cell.

33. The preparation according to claim 25 wherein said microbeads are approximately of a nominal 2 microns in diameter.

34. The preparation according to claim 25 wherein said microbeads comprise at least one of poly(lactide-co-glycolide) (PLG), and polyethelyne glycol (PEG).

35. The preparation according to claim 25 wherein said first DNA sequence codes for a human compatible Fc chain lacking a secretion sequence.

36. The preparation according to claim 25, wherein the antigen for said second DNA sequence is an HIV protein selected from the group consisting of:
a. HIV reverse transcriptase (p51, p66);
b. HIV transcription activator, (tat);
c. HIV internal capsid protein, (p24); and
d. HIV attachment glycoprotiens (Gp41, Gp120).
